(19) United States
(12) Patent Application Publication
(10) Pub. No.: US 2024/0417225 A1
(43) Pub. Date: Dec. 19, 2024

(54) LIFTING COLUMN

(71) Applicant: Ewellix AB, Partille (SE)

(72) Inventors: Marcel Soltermann, Liestal (CH); Martin Vögelin, Liestal (CH); Robert Alexander, Liestal (CH)

(21) Appl. No.: 18/645,979

(22) Filed: Apr. 25, 2024

(30) Foreign Application Priority Data
Jun. 14, 2023 (DE) 10 2023 115 564.4

(51) Int. Cl. B66F 3/10

(52) U.S. Cl. CPC B66F 3/10

(57) ABSTRACT

A lifting column has five column elements that are arranged in a nested manner, at least in portions, as well as a first screw drive for the movement of the three innermost column elements relative to one another and a second screw drive for the movement of the three outermost column elements relative to one another. A coupling tube arrangement is provided which operatively connects the first screw drive with the second screw drive.

The present invention relates to a lifting column.

Lifting columns are known for lifting loads, which lifting columns normally have two or more column elements arranged in a nested manner. In order to telescope the lifting column, i.e. to move the column elements counter to one another, at least one motor arranged in the interior of the lifting column is normally provided. This motor normally drives a worm gear from a threaded spindle and a spindle nut. The lift of the lifting column is restricted by the lift of the spindle nut on the threaded spindle. In order to expand the lift, telescopable threaded spindles or cable pull systems coupled to the worm gear can be used.

Alternatively, two screw drives are also used which are driven by in each case one motor.

One object of the invention is to indicate an improved lifting column. One particular object of the invention is to indicate a lifting column which has a large degree of lift, can be installed with a low degree of outlay and has synchronously movable column elements.

This object is achieved by a lifting column as claimed in the independent claim.

Preferred embodiments are the subject matter of the dependent claims and the following description.

A first aspect of the invention relates to a lifting column which has five column elements arranged in a nested manner at least in portions as well as a first screw drive for the movement of the three innermost column elements relative to one another and a second screw drive for the movement of the three outermost column elements relative to one another. A coupling tube arrangement which operatively connects the first screw drive to the second screw drive is furthermore provided.

An operative connection in the sense of the invention is preferably a, in particular mechanical, coupling. The coupling tube arrangement is consequently at least part of an operative connection between the first and the second screw drive, i.e. the first and second screw drive are operatively connected to one another via the coupling tube arrangement. An operative connection is then also expediently present if a transmission and/or a drive arrangement is/are interconnected, i.e., for example, a drive drives one of the screw drives and the coupling tube arrangement directly or indirectly, for example, via (in each case) a coupling transmission.

A screw drive in the sense of the invention is preferably an assembly for converting a rotational movement into a translational movement. A screw drive is also referred to as a worm gear and expediently has at least one threaded spindle and a threaded spindle nut which runs on an external thread of the threaded spindle. The threaded spindle is preferably screwed directly into the threaded spindle nut. A screw drive can, however, alternatively also involve a ball screw drive or a planetary screw drive in the case of which rolling elements such as balls or planetary rollers roll between the threaded spindle and threaded spindle nut. A screw drive in the sense of the invention can alternatively or additionally also have a hollow spindle and a corresponding hollow spindle nut.

One aspect of the invention is based on the approach of synchronizing two screw drives of a lifting column, which serve in each case to generate a relative movement of in each case three column elements relative to one another, by means of a coupling tube arrangement. As a result of this, only one single drive arrangement is required to extend or retract the lifting column. The lifting column can corresponding be configured in a structurally simpler manner than lifting columns with a cable pull system. A damping of the column elements can furthermore also be dispensed with. In particular, components can thus be omitted. This not only involves simplification of manufacture, but also a reduction in costs.

A flexible configuration of the lifting column is furthermore possible. The drive arrangement can, for example, be operatively connected to the first or second screw drive or to the coupling tube arrangement. The drive arrangement can therefore as required be arranged in any desired one of the column elements, in particular in the outermost, second-outermost, central or second-innermost column element.

An arrangement in a specific column element in the sense of the invention is preferably an arrangement on a base or a top of the corresponding column element. An arrangement in a column element is therefore in particular an arrangement in this column element in any operating state. For example, a drive arrangement arranged in the innermost column element in the retracted state of the lifting column can indeed also be, literally, arranged at the same time in the column elements which lie further to the outside; in the extended state, this drive arrangement is then, however, arranged solely in the innermost column element.

A reliable and at the same time installation space-saving operative connection of the first and second screw drive is enabled by a parallel arrangement of a coupling tube of the coupling tube arrangement to the first and second screw drive. The coupling tube is expediently rotatable within the lifting column, for example, mounted rotatably on the third column element.

The coupling tube arrangement is expediently provided to transmit torques from one column element to another column element, in particular beyond at least one of the column elements, for example, the central column element.

I.e. that at one of the column elements, for example, at the second-innermost column element, a torque can be transmitted to the coupling tube and is provided in another of the column elements, for example, the second-outermost column element. This is expediently possible irrespective of the extent to which the lifting column is telescoped, i.e. how far the column elements are displaced relative to one another. As a result of the coupling tube arrangement, an operative connection which has a particularly small number of components and is mechanically robust can thus be realized between the first and second screw drive.

Preferred embodiments of the invention and their further developments are described below, which in each case, unless not expressly ruled out, can be combined with one another as desired as well as with the aspects of the invention described below.

A space-saving configuration can be achieved by virtue of the fact that the first and second screw drives have in each case a telescopic spindle with an internal threaded spindle and an external hollow spindle. A threaded spindle is preferably a shaft with an external thread or helically running channels for guiding rolling elements. A hollow spindle is correspondingly preferably a hollow shaft with an external thread or helically running channels for guiding rolling elements. In addition to the space-saving formation of the first and second screw drive, as a result of this, a very reliable and highly precise moving of the innermost and outermost column elements relative to one another is also possible.

For synchronous movement of the three outermost or innermost column elements relative to one another, a first hollow spindle of the first screw drive is preferably rotatably mounted on the second-innermost column element. Alternatively or additionally, a second hollow spindle of the second screw drive is rotatably mounted on the second-outermost column element. The hollow spindles are expediently operatively connected via the coupling tube arrangement. I.e. that a rotation of the first hollow spindle can be transmitted by means of the coupling tube arrangement to the second hollow spindle or vice versa. The synchronous movement of the column elements which can be achieved in this manner relative to one another is felt to be aesthetically pleasing by users.

A rotational mounting of the hollow spindles on the second-innermost or second-outermost column element preferably comprises an axial fixing of the hollow spindles on the corresponding column element.

In the case of such a rotatable mounting of the hollow spindles, an extension or retraction of the respective threaded spindles can be achieved by virtue of the fact that the hollow spindles have in each case an internal threaded spindle nut. The threaded spindle nuts can be mounted in a rotationally conjoint manner on the hollow spindles or formed in one piece with these. The threaded spindle nuts are expediently operatively connected to the respective threaded spindle, for example, through engagement of corresponding threads or via corresponding rolling elements. The threaded spindles are expediently arranged rotatably with respect to the respective hollow spindle (and the threaded spindle nut connected in a rotationally conjoint manner thereto), for example, in a rotationally conjoint manner on the central column element or on the outermost column element. The threaded spindles are expediently fixed axially on the central or outermost column element, for example, connected rigidly thereto. This has the advantage that a transmission of force from the screw drives to the column elements does not have to be performed via bearings for mounting of screw drive components, in particular the hollow spindles and threaded spindles. As a result, small rolling bearings or even only plain bearings can also be used for rotational mounting of the screw drive components.

It is, however, also conceivable to mount the threaded spindles rotatably on the central and outermost column element. In this case, the screw drives preferably have in each case a spindle driver, expediently for transmission of torques between the hollow spindles and threaded spindles. For example, in each case a spindle driver can be mounted in a rotationally conjoint manner on the threaded spindles. The hollow spindles expediently have spindle inner profiles on which the spindle drivers are mounted in a rotationally conjoint and axially movable manner.

A profile in the sense of the invention is preferably a configuration of a surface of a substantially cylindrical body which prevents a movement of a component formed to be complementary to the surface and bearing at least in portions on the surface in the circumferential direction, but allows an axial movement. In principle, an arrangement externally or internally on the body is conceivable. An outer or inner profile can correspondingly be discussed.

A torque can be transmitted from one of the column elements to another column element by means of the spindle drivers, in a similar manner to by means of the coupling tube arrangement. It is thus, for example, conceivable to drive the second threaded spindle mounted rotatably on the outermost column element by means of the drive arrangement and transmit the corresponding torque by means of the assigned spindle driver to the second hollow spindle mounted rotatably on the second-outermost column element. The torque can be transmitted further to the first threaded spindle mounted rotatably on the central column element and further by means of the assigned spindle driver to the first hollow spindle as a result of the operative connection of the screw drives via the coupling tube arrangement.

A particularly efficient arrangement, which can be mounted with a particularly low degree of outlay and is installation space-saving, of the coupling tube arrangement in the lifting column can be achieved by virtue of the fact that the coupling tube is mounted rotatably on the central column element. A mechanical operative connection which is substantially free of axial play or lost motion between the first and second screw drive beyond the central column element is thus possible.

It is also conceivable here that two screw drives of identical design are coupled. As a result of this, a further saving in terms of outlay and thus costs is possible if the first screw drive and the second screw drive are of identical design. The hollow and threaded spindles of the screw drives can thus both be formed clockwise.

Alternatively or additionally, the coupling tube arrangement can also have a tube inner profile on which an inner tube driver is mounted in a rotationally conjoint and axially movable manner. The inner tube driver expediently serves here to transmit torques between the coupling tube and the second screw drive, in particular the second hollow spindle, or between the coupling tube and a shaft which can be driven by a motor. If the coupling tube is driven by a motor, its rotation can thus be transmitted to the second hollow spindle. If, however, the shaft or the second threaded spindle is driven by a motor, the rotation of the second hollow spindle or the shaft can be transmitted to the coupling tube.

The shaft which can be driven by a motor is expediently part of the coupling tube arrangement. It is preferably mounted in an axially movable manner in the coupling tube by means of the inner tube driver and connected to the coupling tube in a rotationally conjoint manner. The inner tube driver is preferably mounted on the shaft in a rotationally conjoint manner. Alternatively, the inner tube driver can, however, also be formed in one piece with the shaft. It is in particular conceivable that the shaft has at least in portions a shaft outer profile which is formed in a complementary manner to the tube inner profile.

The shaft is preferably rotationally mounted in a column element different from the central one, for example, on the second-outermost column element. The drive arrangement in this case is correspondingly preferably likewise arranged in the second-outermost column element.

A telescopable coupling tube arrangement can be formed as a result of the provision of the shaft. As a result of this, the coupling tube does not have to be arranged concentrically with the second screw drive, in particular the second hollow spindle. This allows a certain degree of freedom in the arrangement of the first and second screw drive and of the coupling tube arrangement in the lifting column.

Alternatively, the second screw drive, in particular the second hollow spindle, can also be operatively connected, in particular connected in a rotationally conjoint manner, to the coupling tube via the inner tube driver. The inner tube driver can be arranged in a rotationally conjoint manner on the second hollow spindle. The coupling tube is expediently arranged coaxially with the second screw drive, in particular the second hollow spindle. The enables a particularly installation space-saving arrangement of the coupling tube arrangement.

The same advantage can be achieved if the inner tube driver, in one alternative embodiment, is part of the second screw drive, in particular the second hollow spindle. The inner tube driver and the second hollow spindle are expediently formed in one piece. In particular, the hollow spindle can have at least in portions a hollow spindle outer profile which is formed in a complementary manner to the tube inner profile.

To enable the transmission of a rotational movement by means of the coupling tube arrangement beyond a column element, in particular from the second-outermost into the second-innermost column element, the coupling tube preferably has a tube outer profile on which an outer tube driver is mounted in a rotationally conjoint and axially movable manner. The outer tube driver is expediently part of the coupling tube arrangement and is preferably operatively connected to the first screw drive, in particular the first hollow spindle or the first threaded spindle.

A second aspect of the invention relates to an X-ray system with an X-ray source, an X-ray detector and a lifting column according to the first aspect of the invention. The X-ray source or the X-ray detector is expediently mounted on the lifting column. Components of the system can be positioned at a short distance from a top or a base by means of the lifting column. The components can thus be moved precisely relative to one another and indeed independently of whether it is pulled on or pushed for the purpose of positioning.

As an alternative to use in an X-ray system, the lifting column according to the first aspect of the invention can also be used in automation systems, i.e. as part of one or more robots. It is also conceivable to use the lifting column in a, possibly mobile, goods or passenger lift.

The invention is explained in greater detail below on the basis of figures. Where expedient, elements with the same effect are provided with identical reference signs herein. The invention is not restricted to the exemplary embodiments represented in the figures, also not in relation to the functional features. The previous description and the following description of the figures contain numerous features which are reproduced in the dependent subordinate claims partially combined to several. The person skilled in the art will, however, also consider these features and all of the other features disclosed above and in the following description of the features in each case individually and join them together to form expedient further combinations. In particular, all of the stated features can be combined in each case individually and in any desired suitable combination with the lifting column according to the first aspect of the invention and the X-ray system according to the second aspect of the invention.

At least partially schematically:

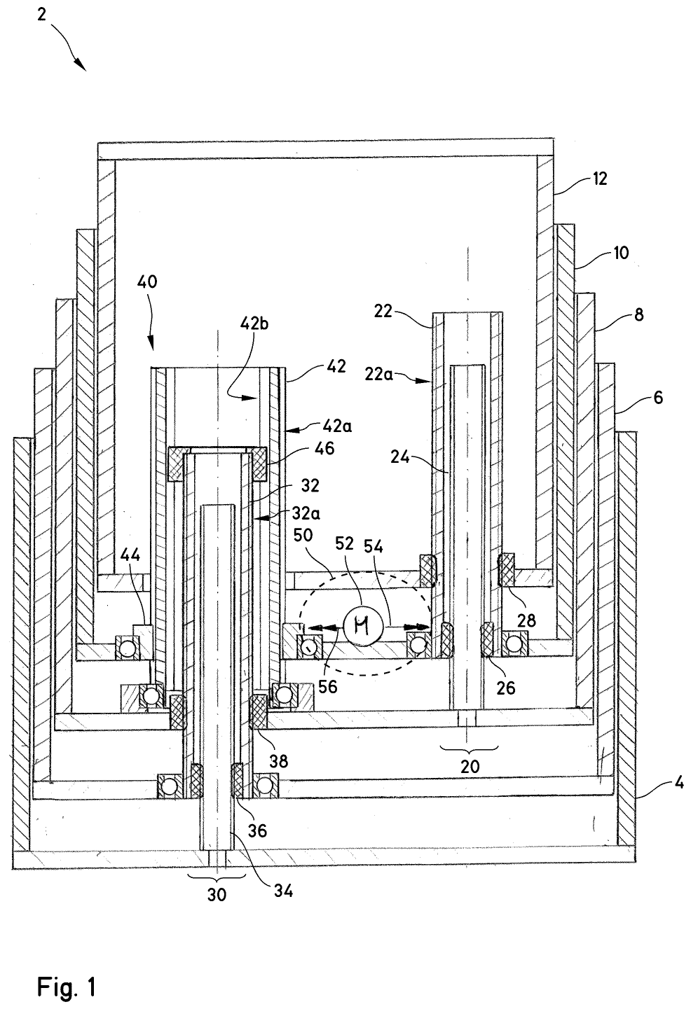

FIG. 1 shows a first example of a lifting column;

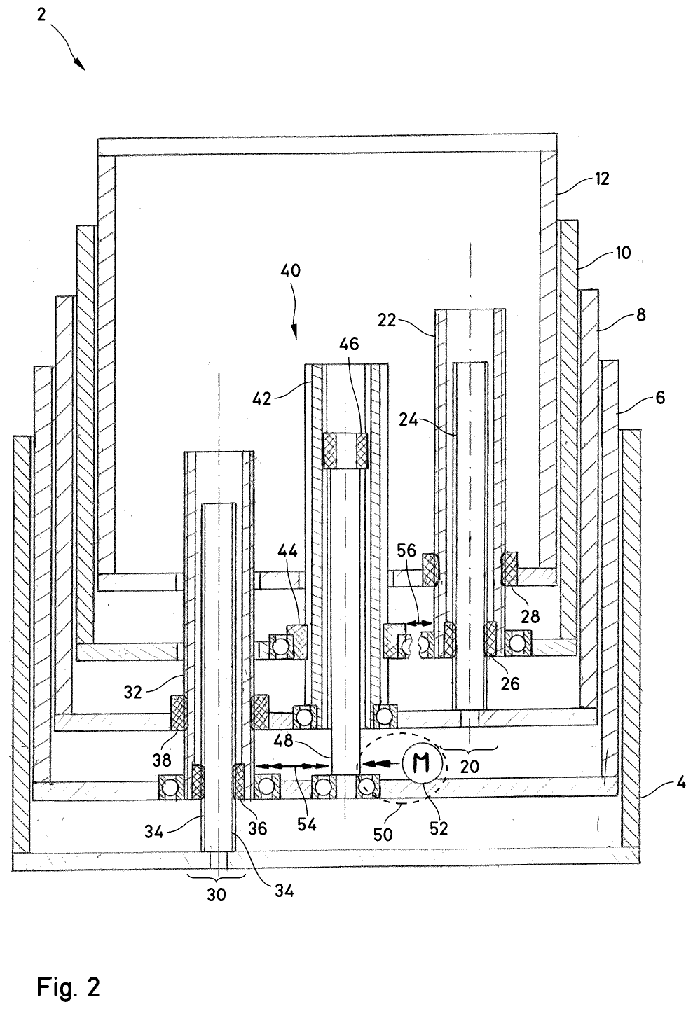

FIG. 2 shows a second example of a lifting column;

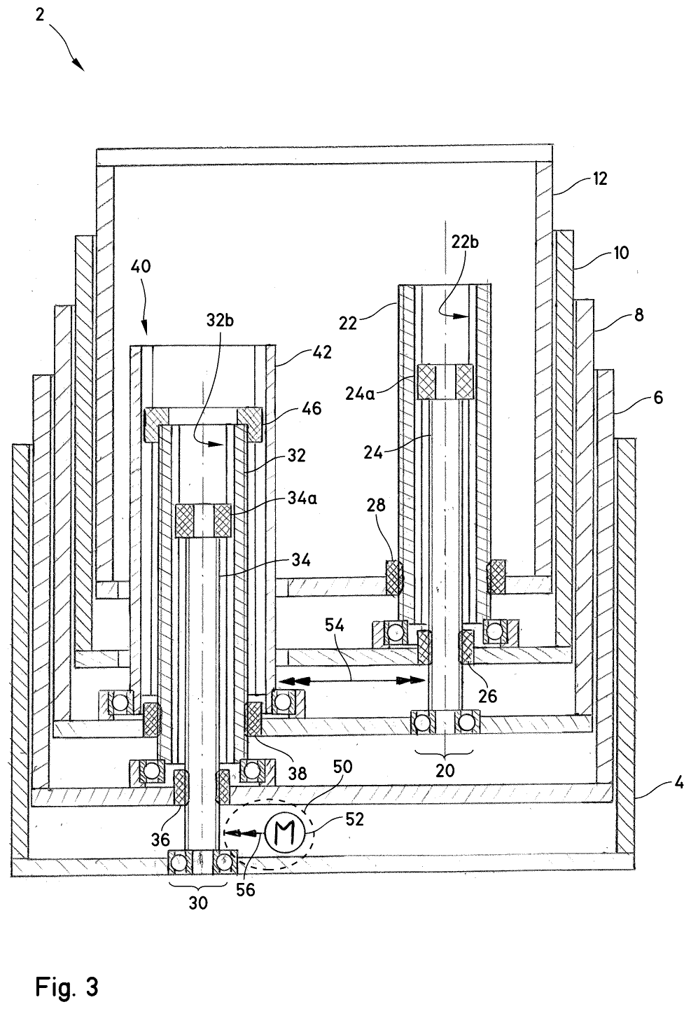

FIG. 3 shows a third example of a lifting column; and

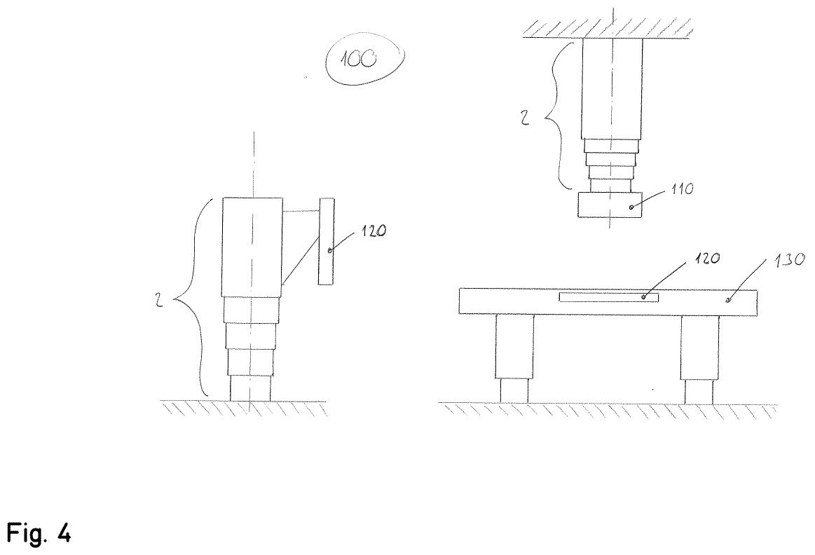

FIG. 4 shows an example of an X-ray system.

FIG. 1 shows a first example of a lifting column 2 with five column elements 4, 6, 8, 10, 12 arranged in a nested manner at least in portions. In particular, a second-outermost column element 6 is arranged at least in portions in an outermost column element 4, a central column element 8 at least in portions in the second-outermost column element 6, a second-innermost column element 10 at least in portions in the central column element 8 and an innermost column element 12 at least in portions in the second-innermost column element 10. The column elements 4, 6, 8, 10, 12 are rotationally conjoint relative to one another.

The column elements 4, 6, 8, 10, 12 can be moved axially relative to one another by means of a first screw drive 20 and a second screw drive 30. The two screw drives 20, 30 expediently operate in a complementary manner to one another.

I.e. that the first screw drive 20 can move the three innermost column elements 8, 10, 12 relative to one another and the second screw drive 30 can move the three outermost column elements 4, 6, 8 relative to one another. As a result of this, it is possible to increase the lift in comparison with conventional lifting columns or achieve at least a comparable lift with a significantly more robust design.

A coupling tube arrangement 40 operatively connects the first screw drive 20 to the second screw drive 30. This means that the coupling tube arrangement 40 is part of an operative connection between the first and the second screw drive 20, 30. In the present case, this operative connection additionally comprises a drive arrangement 50. The drive arrangement 50 has a motor 52 and optionally a first and/or second transmission 54, 56 for transmitting a torque generated by the motor 52 to the first screw drive 20 or the coupling tube arrangement 40.

The first screw drive 20 has a first hollow spindle 22 and a first threaded spindle 24 arranged at least in portions therein. The first hollow spindle 22 can be directly driven by the motor 52, in particular via the first transmission 54. The first hollow spindle has on the inside a first threaded spindle nut 26 into which the first threaded spindle 24 can be screwed.

The second screw drive 30 is formed to be substantially of the same design as the first screw drive 20. It has a second hollow spindle 32 and a second threaded spindle 34 arranged therein at least in portions. The second hollow spindle can be indirectly driven by the motor 52, namely via the coupling tube arrangement 40. The coupling tube arrangement 40 can optionally be operatively connected via a second transmission 56 to the motor 52. The second hollow spindle 32 has on the inside a second threaded spindle nut 36 into which the second threaded spindle 34 is screwed.

For synchronous movement of the three innermost column elements 8, 10, 12 relative to one another, the first hollow spindle 22 is rotatably mounted on the second-innermost column element 10 and axially fixed thereon. The first threaded spindle 24 is mounted in a rotationally conjoint manner on the central column element 8. A first hollow spindle nut 28 is mounted in a rotationally conjoint manner on the innermost column element 12, into which hollow spindle nut 28 the first hollow spindle 22 is screwed with an external thread 22a.

As a result of this, the first hollow spindle nut 28, and thus the innermost column element 12, moves, in the case of a rotation of the first hollow spindle 22 driven by the motor 52 axially along the first hollow spindle 22, and thus relative to the second-innermost column element 10, while the first threaded spindle 24 extends or retracts from the first hollow spindle 22 and as a result moves the central column element 8 relative to the second-innermost column element 10.

For synchronous movement of the three outermost column elements 4, 6, 8 relative to one another, the coupling tube arrangement 40 has a coupling tube 42 arranged coaxially with the second screw drive 30, in particular the second hollow spindle 32. The coupling tube 42 has a tube outer profile 42a on which an outer tube driver 44 is mounted in a rotationally conjoint and axially movable manner. The outer tube driver 44 thus enables the transmission of a torque to the coupling tube 42. The outer tube driver 44 is operatively connected to the motor 52, in particular via the second transmission 56.

The coupling tube 42 also has a tube inner profile 42b on which an inner tube driver 46 is mounted in a rotationally conjoint and axially movable manner. The inner tube driver 46 is mounted on the second hollow spindle 32 in a rotationally conjoint manner. As a result of this, the coupling tube 42 can transmit a rotation driven by the motor 52 to the second hollow spindle 32.

The coupling tube 42 is rotationally mounted on the central column element 8, while the second hollow spindle 32 is rotationally mounted on the second-outermost column element 6. The coupling tube arrangement 40 thus serves to transmit a torque from the second-innermost column element 10 beyond the central column element 8 to the second-outermost column element 6.

A second hollow spindle nut 38 is mounted in a rotationally conjoint manner on the central column element 8, into which hollow spindle nut 38 the second hollow spindle 32 is screwed with an external thread 32a. The second threaded spindle 34 is mounted in a rotationally conjoint manner on the outermost column element 4.

As a result of this, the second hollow spindle nut 38, and thus the central column element 8, moves, in the case of a rotation of the second hollow spindle 32 driven by the motor 52 and transmitted by the coupling tube arrangement 40 axially along the second hollow spindle 32, and thus relative to the second-outermost column element 6, while the second threaded spindle 34 extends or retracts from the second hollow spindle 34 and as a result moves the outermost column element 4 relative to the second-outermost column element 6.

As an alternative to the arrangement shown of the drive arrangement 50 in the second-innermost 10, an arrangement in the second-outermost column element 6 is also conceivable. The motor 52 is then operatively connected directly to the second hollow spindle 32, optionally via the first transmission 54. The second hollow spindle 32 can transmit a torque applied as a result of it via the inner tube driver 46 and the coupling tube 42 to the outer tube driver 44. The outer tube driver 44 is expediently operatively connected to the first hollow spindle 22, for example, via the second transmission 56.

The tube outer profile 42a and/or the tube inner profile 42b can be formed by a non-round outer or inner contour of the coupling tube 42 in the circumferential direction. The coupling tube 42 has this non-round outer contour expediently at least over a majority of its length. The coupling tube 42 expediently does not have a non-round outer contour in the region of its rotational mounting on the central column element 8. A non-round inner or outer contour can be formed, for example, by an oval or angular outer or inner form in cross section of the coupling tube 42. Axially running grooves or webs in or on which complementary webs or grooves of the tube drivers 44, 46 sit or engage, are alternatively conceivable. Other configurations are, however, also in principle conceivable.

A load (not represented) can be carried with the lifting column 2. In the case of the lifting column 2 shown in FIG. 1, it has been shown to be advantageous that the load is not supported via the bearings for mounting the components of the first and second screw drive 20, 30 and the coupling tube arrangement 40.

Instead, a flow of force brought about by such a load will be supported exclusively via the first and second hollow spindles 22, 32, the first and second threaded spindles 24, 34 and the nuts 26, 28, 36, 38.

FIG. 2 shows a second example of a lifting column 2 with five column elements 4, 6, 8, 10, 12 arranged in a nested manner at least in portions and axially movable relative to one another by means of a first and second screw drive 20, 30. The lifting column 2 differs from the lifting column shown in FIG. 1 in that the drive arrangement 50 is arranged in the second-outermost column element 6 and the coupling tube arrangement 40, in particular the coupling tube 42, is not arranged coaxially with the second screw drive 30.

Instead, the coupling tube arrangement 40 is arranged separately from the first and second screw drive 20, 30 and is operatively connected thereto via the first and second transmission 54, 56.

In particular, the coupling tube arrangement 40 has a shaft 48 which is rotationally mounted on the second-outermost column element 6. The shaft 48 can be driven by means of the motor 52 and is operatively connected via the first transmission 54 to the second hollow spindle 32. The inner tube driver 46 is mounted in a rotationally conjoint manner on the shaft 48 or is formed in one piece therewith so that a rotation, driven by the motor 52, of the shaft 48 is transmitted to the coupling tube 42. The outer tube driver 44 is operatively connected to the first hollow spindle 22, in particular via the second transmission 56. As a result of this, a torque can be transmitted from the second-outermost column element 6 via the central column element 8 up to the second-innermost column element 10.

As a result of the separate arrangement of the coupling tube arrangement 40, a particularly efficient arrangement of the coupling tube arrangement 40 together with the first and second screw drive 20, 30 within the lifting column 2 is possible. The first and second screw drive 20, 30 and the coupling tube arrangement 40 can be mounted, for example, in a particularly installation space-saving triangular arrangement.

In this variant too, a load is not supported by the bearings for mounting the screw drives 20, 30.

FIG. 3 shows a third example of a lifting column 2 with five column elements 4, 6, 8, 10, 12 arranged in a nested manner at least in portions and movable relative to one another by means of a first and second screw drive 20, 30. In contrast to the examples shown in FIGS. 1 and 2, the threaded spindles 24, 34 have in each case a spindle driver 24a, 34a which is mounted in a rotationally conjoint manner on the threaded spindles 24, 34 or can be formed in one piece therewith. The spindle drivers 24a, 34a are mounted on a spindle inner profile 22b, 32b of the first or second hollow spindle 22, 32 in a rotationally conjoint and axially movable manner. The hollow spindles 22, 32 are therefore not operatively connected via a threaded spindle nut arranged internally therein, mounted in a rotationally conjoint manner or formed in one piece, but rather via the spindle drivers 24a, 34a to the respective threaded spindle 24, 34, in particular connected in a rotationally conjoint manner.

The threaded spindles 24, 34 are rotationally mounted on the central or outermost column element 8, 4. The second threaded spindle 34 is operatively connected directly to the drive arrangement 50 arranged likewise in the outermost column element 4.

The first threaded spindle 24 is operatively connected via a first transmission 54 to the coupling tube 42 in the central column element 8. Since both the first threaded spindle 24 and the coupling tube 42 are rotationally mounted on the central column element 8, and thus axially fixed thereto, an outer tube driver is not necessary.

For synchronous movement of the column elements 4, 6, 8, 10, 12 relative to one another, the threaded spindles 24, 34 are screwed into threaded spindle nuts 26, 36 mounted in a rotationally conjoint manner on the second-innermost or second-outermost column element 10, 6. In the case of rotation of the threaded spindles 24, 34, the second-innermost column element 10 is consequently moved relative to the central column element 8 and the second-outermost column element 6 is moved relative to the outermost column element 4.

The hollow spindles 22, 32 are screwed into hollow spindle nuts 28, 38 mounted in a rotationally conjoint manner on the central or innermost column element 8, 12 so that, in the event of rotation of the hollow spindles 22, 32, the innermost column element 12 is moved relative to the second-innermost column element 10 and the central column element 8 relative to the second-outermost column element 6.

The torque introduced by the motor 52 is consequently transmitted by means of the second screw drive 30 from the outermost column element 4 into the second-outermost column element 6, by means of the coupling tube arrangement 40 from the second-outermost column element 6 into the central column element 8 and by means of the first screw drive 20 from the central column element 8 into the second-innermost column element 10.

In the example shown in FIG. 3, a flow of force runs, in contrast to the examples shown in FIGS. 1 and 2, through the bearings of the hollow spindles 22, 32 and threaded spindles 24, 34. For this purpose, the drive arrangement 50, in particular the motor 52, can be arranged in the outermost column element 4. This can facilitate the mounting of the lifting column 2 and/or the energy supply of the motor 52, for example, if the lifting column 2 is used as a floor stand or ceiling mount and the outermost column element 4 stands on the ground or the outermost column element 4 is attached to the ceiling.

FIG. 4 shows an example of an X-ray room in which an X-ray system 100 with an X-ray source 110 and two X-ray detectors 120 as well as two lifting columns 2, which can be telescoped in particular five times, is arranged. Here, one X-ray detector 120 is integrated into a patient table 130, while the other X-ray detector 120 is part of a profile wall unit and is height-adjustable by means of one of the lifting columns 2. The X-ray source 110 is likewise mounted in a height-adjustable manner by means of the other lifting column 2.

The lifting columns 2 can in this regard generally serve as a stand, namely in particular a floor stand and/or as a ceiling mount.

### LIST OF REFERENCE SIGNS

2 Lifting column
4 Outermost column element
6 Second-outermost column element
8 Central column element
10 Second-innermost column element
12 Innermost column element
20 First screw drive
22 First hollow spindle
22a External thread
22b Spindle inner profile
24 First threaded spindle
24a First spindle driver
26 First threaded spindle nut
28 First hollow spindle nut
30 Second screw drive
32 Second hollow spindle
32a External thread
32b Spindle inner profile
34 Second threaded spindle
34a Second spindle driver
36 Second threaded spindle nut
38 Second hollow spindle nut
40 Coupling tube arrangement
42 Coupling tube
42a Tube outer profile
42b Tube inner profile
44 External tube driver
46 Inner tube driver
48 Shaft
50 Drive arrangement
52 Motor
54 First transmission
56 Second transmission
100 X-ray system
110 X-ray source
120 X-ray detector
130 Patient table

1-11. (canceled)

12. A lifting column, comprising:
five column elements arranged in a nested manner at least in portions, said five column elements being disposed as three innermost column elements and three outermost column elements;
a first screw drive configured for moving said three innermost column elements relative to one another;
a second screw drive configured for moving said three outermost column elements relative to one another; and
a coupling tube arrangement operatively connecting said first screw drive with said second screw drive.

13. The lifting column according to claim 12, wherein each of said first and second screw drives has a telescopic spindle with an internal threaded spindle and an external hollow spindle.

14. The lifting column according to claim 13, wherein a first hollow spindle of said first screw drive is rotatably mounted on a second-innermost column element of said five column elements, and a second hollow spindle of said second screw drive is rotatably mounted on a second-outermost column element of said five column elements.

15. The lifting column according to claim 13, wherein each of said hollow spindles has an internal threaded spindle nut which is operatively connected to a respective said threaded spindle.

16. The lifting column according to claim 13, wherein said threaded spindles have a spindle driver and said hollow spindles have spindle inner profiles on which said spindle drivers are mounted rotationally conjointly and axially movably.

17. The lifting column according to claim 12, wherein a coupling tube of said coupling tube arrangement is rotatably mounted on a central column element of said five column elements.

18. The lifting column according to claim 12, wherein said first screw drive and said second screw drive are identically designed screw drives.

19. The lifting column according to claim 12, wherein said coupling tube arrangement has an inner tube driver which is mounted on a tube inner profile of said coupling tube rotationally conjointly and axially movably.

20. The lifting column according to claim 19, wherein said coupling tube arrangement has a shaft to be driven by a motor, which is mounted axially movably in said coupling tube by way of said inner tube driver and which is rotationally conjointly connected to said coupling tube.

21. The lifting column according to claim 19, wherein said second screw drive is operatively connected to said coupling tube via said inner tube driver.

22. The lifting column according to claim 12, wherein said coupling tube arrangement has an outer tube driver which is rotationally conjointly and axially movably mounted on a tube outer profile of said coupling tube.
